(19) United States
(12) Patent Application Publication
(10) Pub. No.: US 2014/0182620 A1
(43) Pub. Date: Jul. 3, 2014

(54) USES FOR AN ACETAL BASED COMPOSITION AND METHOD OF THINNING/CLEANING/STRIPPING

(71) Applicant: WOCEB BVBA, Londerzeel (BE)

(72) Inventor: Bruce Lanhove, Londerzeel (BE)

(73) Assignee: Woceb bvba, Londerzeel (BE)

(21) Appl. No.: 13/863,668

(22) Filed: Apr. 16, 2013

(30) Foreign Application Priority Data
Jan. 3, 2013 (BE) 2013/0003

(51) Int. Cl. C07C 43/315

(52) U.S. Cl. CPC C07C 43/315
USPC 134/2; 568/601; 428/35.7

(57) ABSTRACT

An acetal-based composition is described which includes an acetal of formula R—O—CH₂—O—R′. R and R′ are mutually independent alkyl or alkoxyalkyl substituents with at least three carbon atoms. The composition is useful as a thinner and/or a detergent and/or a stripper. The composition may be used to clean surfaces which are metal, glass or wood and is particularly well adapted for use in the automotive industry. The thinning/cleaning/stripping composition may be stored in a polyethylene container.

### TECHNICAL FIELD

The invention refers to an eco-friendly and safe thinner and detergent based on a non-volatile acetal. The product can be used for the replacement of thinner and other harmful solvents as a thinner for paint and/or detergent for the removal of residues, e.g. adhesive, resin, ink. This product can be applied, amongst others, in the automotive industry, whereby car body workshops can be made thinner-free.

### STATE OF THE ART

Aromatic solvents, e.g. toluene, are well-known in the industry for use in compositions for the degreasing of surfaces, as paint thinner, stain remover or a general detergent. Typically, the preferred characteristics of these aromatic solvents are their solubility, short drying times, while little or no residue is left on the treated surface. A problem with the known thinners/detergents based on aromatic solvents is their high volatility and low flash-point. This may lead to problems with regards to flammability, combustibility and safety for the users.

There are alternatives like chlorinated solvents e.g. dichloromethane, dichloroethene, trichloroethene. These substances are very volatile. For their use very good aspiration systems and ventilation is required. Another problem with chlorinated solvents for use in cleaning products and/or thinners are the chlorinated residues. Also, the presence of chlorine may lead to corrosion issues.

Although solvents such as dimethyl formamide, dimethyl acetamide, N-methyl pyrrolidone, provide efficient cleaners, their toxicological characteristics pose a threat to the health.

In earlier times, a combination of dangerous chemical paint removers and the mechanical and manual polishing of the paint from the surface of the vehicle was a common practice for the removal of paint from cars. Environmental issues and the health of employees related to the use of chemical removers are well-known. This process is mostly substituted by a polishing system. No adequate solution has been found for the issue of the removal of paint or mastic from paint material, such as spray guns.

Enhanced or alternative compositions for use as detergents and/or thinners are required. More specifically, the use of alternatives should provide a solution which is safe, healthy and eco-friendly, while their effectiveness should be maintained. Besides, the alternative should preferably be readily available and cost efficient.

The present invention has the objective to find a solution for at least one of the previous problems.

### SUMMARY OF THE INVENTION

The invention thereto provides the use of a composition based on non-volatile acetals, as described in claim 1.

In a second aspect, the invention provides a method for the thinning and/or cleaning and/or stripping by means of an acetal based product, as described in claim 9.

In a third aspect, the invention provides a container suitable of encompassing the composition, as described in claim 15.

Further improvements are represented in the dependent claims.

### DETAILED DESCRIPTION

Unless otherwise defined, all terms used in the description of the invention, also the technical and scientific terms, shall mean what is generally understood by an expert in the technical area of the invention. For a better evaluation of the description, a specific explanation is provided for the following terms.

“A”, “an”, “the” in this document refer both to the singular and the plural, unless otherwise can be understood from the context. For instance “a segment” means one or more than one segment.

When “approximately” or “round” is stated for a measurable unit, parameter, time period or moment, the variations shall be +/−20% or less, preferably +/−10% or less, more preferably +/−5% or less, even more preferably of +/−1% or less, and still even more preferably of +/−0.1% or less than and from the said value, provided such variations apply to the described invention. Nevertheless, it should be understood here that also an explicit specification is made of the unit value itself for which the term “approximately” or “round” has been used.

The terms “comprise”, “comprising”, “consist of”, “consisting of”, “provided with”, “contain”, “containing”, “include”, “including”, “concern”, “concerning” are synonyms and concern inclusive or open terms describing what follows, without any exclusion or limitation of the existence of any other components, characteristics, elements, parts, steps, which are known from or described in the state of the art.

The quotation of numerical intervals by their endpoints comprises all integers, fractions and/or real numbers between and including these endpoints.

Firstly, the invention concerns the use of a composition comprising an acetal with formula R—O—CH2-O—R′, whereby R and R′ are mutually independent alkyl or alkoxyalkyl substituents with at least three carbon atoms, as a thinner and/or a detergent and/or a stripper.

The term “acetal” in this invention means a compound with the formula R—O—CH2-O—R′, for instance obtained by the reaction of aldehydes and alcohols in the presence of a catalyst and after the segregation of the reactive products, water and acetal. A process for making acetals is described in WO 00/09575. The aldehyde in the present invention is formaldehyde. Alcohols may be propanol, butanol.

CH3(CH2)2-O—CH2-O—(CH2)2-CH3 propylal

CH3(CH2)3-O—CH2-O—(CH2)3-CH3 butylal

The groups R and R′ may be different or identical. Preferably, R is equal to R′.

The groups R and R′ may be an alkyl or alkoxyalkyl substituents. In the present invention the R and R′ substituents comprise at least three carbon atoms.

Consequently, the acetals have a flash-point which does not classify the product as flammable.

The term “flash-point” means the minimum temperature at which the headspace of a material, such as a liquid, is ignited by a flame under specific standard conditions. The lower the flash-point is the higher the probability that the material can be ignited. The higher the flash-point the safer the product is to work with.

The inventor(s) found out that said acetals can be implemented as thinner and/or detergent and/or stripper. Specifically, said composition can be implemented for thinning paint, removing spots and stains and/or as a general remover of paint from tools, equipment and other surfaces in a paint process.

By the term “thinner” or “thinning of” is meant the dilution of a composition, such as paint, without having a substantial influence on other physical and chemical properties. Examples of such properties for paint are colour, luster, hardness, dispersibility.

By the term “cleaning means” or “cleaning of” is meant a composition which has for effect that residue can be removed from a substrate. In a paint process, the paint thinners can be effective for the removal of paint in a wet condition. For instance, the removal of paint from a paint brush, or the absorption of a stain due to spilled paint.

By the term “remover” or “stripper” is meant a cleaning means capable of removing residue in a solid form, for instance dried paint. A remover or stripper is suitable for the removal or stripping of a coating from a substrate. As a synonym the word descaler can be used.

A method for the removal of a coating from a substrate comprises the contacting of the coating with the composition.

Preferably, the composition comprises 50%-100% by weight of an acetal, more preferably 80%-99% by weight of an acetal, most preferably, 90%-98%, expressed with respect to the total weight of the composition.

Preferably, the composition is substantially water and/or acid-free. This means a content of less than 5%, preferably, less than 4%, more preferably, less than 3%, most preferably less than 2% and still more preferably, less than 1%. More preferably, the composition has a content of lower than 5% by weight of water.

In a preferred implementation form of the invention the acetal is a glycol ether formal of formula [CH3O(CH2CH2O)n]2CH2 whereby n=1 or 2, preferably, 1. These acetals are obtained from the reaction of an ethylene glycol monoethyl 97% ether, also called 2-ethoxyethanol, with formaldehyde. A possible synthesis path is described in U.S. Pat. No. 4,093,666.

Most preferably, the acetal is a glycol ether formal with the formula CH₃—O—(CH₂)₂—O—CH₂—O—(CH₂)₂—O—CH₃. This compound has the chemical name 2,5,7,10-tetraoxaundecane, abbreviated as TOU.

TOU is advantageously used in a detergent/stripper solution. A TOU-based detergent/thinner/stripper is not flammable or harmful for the environment and does not evaporate. The flash-point measured according to the closed cup method according to Pensky-Martens is 88° C.; measurement according to the open cup method according to Cleveland provides a flash-point of 92° C. An advantage of TOU is its complete miscibility with water.

In a preferred embodiment of the invention, the used composition comprises 50%-100% by weight of TOU, preferably, 60%-99% by weight of TOU, more preferably, 70%-98% by weight of TOU, most preferably, at least 95% by weight of TOU, expressed with respect to the total weight of the composition.

In a preferred form the used product substantially comprises pure TOU, in other words, at least 95% by weight of TOU, preferably, 96% by weight of TOU, more preferably, 97% by weight of TOU, most preferably, 98% by weight of TOU.

Preferably, the composition is substantially water and/or acid-free.

In a more preferred form the composition has a content of less than 5% by weight of water, preferably, less than 4% by weight of water, more preferably, less than 3% by weight of water, most preferably less than 2% by weight of water. Due to the low presence of water the product is not inactivated and its force is maintained. TOU has a long durability with little water content.

The durability of TOU in an undiluted form with a water content of less than 5%, is at least 3 months, preferably, 6 months, more preferably, 12 months.

In a preferred embodiment, the composition has an acid content lower than 5 weight %, preferably lower than 4 weight %, more preferably lower than 3 weight %, most preferably lower than 2 weight %.

In a preferred embodiment, the composition is used for the descaling of a residue from a substrate. In the descaling the residue is not dissolved, but loses its adherence. This has the advantage that no water is required, the product remains active and can be reused after the removal of the residue. When applied on a grease layer, the grease is sedimented after a rest period of approximately 24 hours. This causes a clean upper layer which can be reused. This contrasts greatly with thinners that are dissolving the whole or part of the residue, and hence are to be disposed of.

The acetal based formulations of the present invention are effective for the removal of different organic polymer materials and organic residues. The implementation of acetals is beneficial for the removal of paint, mastic, adhesive, grease, oil, ink, tar and/or soot.

Therefore, it suits many applications varying between: paint removal at paint companies, car body companies, for private use when painting at home, removal of graffiti; oil removal in the car-repair branch; grease removal, not only for the car-repair branch, but also for metal manufacturing, glass manufacturing, food branch, ink remover for printers, textile manufacturing; adhesive remover for letter-print, floors for instance balaton, metal panels; removal of tar or soot from glass, for instance the scale on heaters, barbecues and chimneys.

Preferably, the substrate is a metal, glass or wooden surface. The inventor(s) have determined that the composition is beneficial for the cleaning/stripping of metal surfaces, preferably, shaped metal articles. The composition is not corrosive. A corrosion inhibitor is not required in the composition. Grease and/or paint residues can be removed from glass or wood without affecting these materials.

Preferably, a composition is used as a stripper and/or thinner in the car industry.

The stripper can be used for the cleaning of spray guns when painting cars. The formulation can be used for the removal of both the wet and the dried form of caked paint.

In a preferred embodiment the acetal based composition is used for the removal of layers on a car body, for instance alkyd primers, alkyd upper layers and acryl nitrocellulose upper layers. It offers an eco-friendly alternative for methylene chloride and toluene based removers. Also, there is no need to invest in expensive aspiration equipment and ventilation. Another advantage is that fire safety measurements, as imposed by aTEX and/or AREI standards, are excluded.

In a more preferred embodiment the composition has a water content of lower than 5 weight %, preferably lower than 4 weight %, more preferably lower than 3 weight %, most preferably lower than 2 weight %.

Various methods can be applied to remove residue from a substrate. Mainly, these consist in contacting the residue covered substrate with the stripper and/or cleaning composition. For example, the substrate containing the residue can be immersed in a strip- and cleaning bath, or the stripper and cleaning composition can be sprayed over the surface of the residue.

In a second aspect, the invention relates to a method for the cleaning of a metal surface, which is at least partially covered with a residue, comprising the step: applying a composition according to an embodiment of the invention, on the metal surface to be cleaned, and removing the residue.

Preferably, the composition is applied with a high pressure device. For small quantities a spray bottle or sprinkler can be used.

In a preferred embodiment of the invention, the method further comprises the step: separating solid residue and acetal. In the removal of solid residue, for instance dried paint on a metal frame, by the action of the stripper, a liquid with residue remnants is formed. As the residue remnants do not dissolve but detach, solid matter and liquid can be separated. The acetal liquid can be reused. When reused, the water content should be taken into account, because addition of water reduces its effectiveness.

Preferably, the acetal and the surface for cleaning, for instance metal, glass, or wood, are in contact during a period of 10 s up to several hours. For wet applications, a few seconds are sufficient. For the removal of dried residues, for instance paint, 1 to several hours are required depending on the thickness and composition. For use as a descaler, by the action of the stripper on dried and solid residues, a longer period can be necessary.

Preferably, the cleaning is performed at a temperature of 20° C.-30° C. It is not necessary to warm the composition, or to perform the treatment at a higher temperature. Room temperature is sufficient.

In a preferred embodiment of the invention, the method further comprises the steps: post-rinsing of the cleaned surface with water and drying of the rinsed surface. Due to the miscibility of acetals and specifically of TOU with water, removal is easy by the addition of water. Simultaneously, the product is inactivated by important quantities of water.

TOU has the advantage that it mixes very well with water. Post-rinsing with water results in dilution of the product and sweeping away by the water. Where TOU itself may leave a greasy film, the post-rinsing with water takes care of the removal of this greasy film. The rinsed surface can be dried in a simple way in the air.

In a final aspect, the invention concerns a container comprising a composition as previously described, whereby the container is made from polyethylene; preferably, high density polyethylene (HDPE).

In a preferred embodiment the packaging is a HDPE container with TOU in a pure form, with less than 5% of water, preferably, less than 3% of water. In comparison, the storage of TOU in a PET bottle affects the material. After one day half of the material was melted. After a second day the bottle leaked like a sieve.

In what follows, the invention is described by means of non-limitative examples illustrating the invention, and which are not intended nor may be interpreted to limit the scope of the invention.

### EXAMPLE 1

A stripping/cleaning solution was prepared by taking 1 liter of 2,5,7,10-tetraoxaundecane, undiluted, degree of purity 97%. Paint was applied on a car body with a paint gun. After the painting process stopped, the paint gun was dismantled. The parts were cleaned with TOU by means of a pressurized cleaning device during a period of a few seconds up to some minutes. After this process the parts were rinsed with water and dried in the open air or with a cloth. A visual inspection was performed on the conduits and metal parts of the spray gun. There were no traces of paint residue.

### EXAMPLE 2

A caked and dried container for the mixing of paint, filled with paint residue, was soaked in a solution during one night, according to example 1. The paint loosened from the container. The lumps of paint were easily separable from the solution. This could be reused.

### EXAMPLE 3

A metal grid tainted with paint, mastic, grease and varnish was sprinkled with 2,5,7,10-tetraoxaundecane (TOU). After two hours the grid was cleaned with cold water from a high pressure cleaning device. The grid was clean without the metal being affected.

### EXAMPLE 4

In this comparative example the window glass of a fireplace was cleaned. On half of the surface a standard cleaning product for fire place window glass was applied (“Rannon 'Tout”). The other half was cleaned with undiluted TOU.

With the first product 90% of the treated surface was cleaned. With TOU, this was 100%

What is claimed is:

1. A composition for use as a as thinner and/or detergent and/or stripper comprising an acetal of formula R—O—CH₂—O—R′, whereby R and R′ are mutually independent an alkyl or alkoxyalkyl substituent with at least three carbon atoms.

2. The composition according to claim 1, whereby the acetal is CH₃—O—(CH₂)₂—O—CH₂—O—(CH₂)₂—O—CH₃.

3. The composition according to claim 1, wherein the composition comprises 50%-100% by weight of the acetal, expressed with respect to the total weight of the composition.

4. The composition of claim 1 which is substantially water and/or acid-free, more specifically with a content lower than 5% by weight.

5. Method for the cleaning and/or stripping of a metal surface at least partially covered with a residue, comprising the steps of:
applying the composition of claim 1, on the metal surface to clean, and
removing the residue.

6. Method according to claim 5, wherein the composition is applied with a high pressure cleaning device.

7. Method according to claim 5, further comprising the step of: separating residue and acetal.

8. Method according to claim 5, wherein the acetal applied and the metal surface to clean are in contact during 10 s to 60 min.

9. Method according to claim 5, wherein the cleaning is performed at a temperature of 20° C.-30° C.

10. Method according to claim 5, further comprising the steps of:
post-rinsing of the cleaned surface with water, and
drying of the rinsed surface.

11. Container comprising the composition according to claim 1, wherein the container is made from polyethylene.

12. Method for descaling of a residue from a substrate at least partially covered with the residue, comprising the steps of:
applying the composition of claim 1, on the substrate to descale, and
removing the residue.

13. The method of claim 12, wherein the residue is paint, adhesive, grease, oil, mastic, and/or ink.

14. The method of claim 12, wherein the substrate is a metal, glass or wooden surface.
